(19) United States
(12) Patent Application Publication
(10) Pub. No.: US 2005/0144665 A1
(43) Pub. Date: Jun. 30, 2005

(54) Recombinase mediated gene traps

(75) Inventors: William J. Gordon-Kamm, Urbandale, IA (US); L. Alexander Lyznik, Johnston, IA (US); Christopher J. Scelonge, Ankeny, IA (US); Theodore M. Klein, Wilmington, DE (US)

Correspondence Address: PIONEER HI-BRED INTERNATIONAL INC., 7100 N.W. 62ND AVENUE, P.O. BOX 1000, JOHNSTON, IA, 50131, US

(73) Assignees: Pioneer Hi-Bred International, Inc.; E.I. du Pont de Nemours and Company

(21) Appl. No.: 11/015,612

(22) Filed: Dec. 17, 2004

Related U.S. Application Data
(60) Provisional application No. 60/530,402, filed on Dec. 17, 2003.

(51) Int. Cl. A01H 1/00; C12N 15/82

(52) U.S. Cl. 800/278; 435/468

(57) ABSTRACT

The present invention provides methods for identifying a genetically modified plant cell comprising a recombinase-mediated exchange between a first nucleotide sequence and a second nucleotide sequence. The present invention provides a method for obtaining a genetically modified plant cell wherein a functional recombination at both the 5′ and 3′ ends of the nucleotide can be identified.

### CROSS REFERENCE TO RELATED APPLICATIONS

This application claims priority to U.S. Ser. No. 60/530,402 filed Dec. 17, 2003 the disclosure of which is incorporated herein by reference.

### TECHNICAL FIELD

The present invention relates generally to plant molecular biology and plant transformation.

### BACKGROUND OF THE INVENTION

Genetic modification techniques enable one to insert exogenous nucleotide sequences into an organism's genome. A number of methods have been described for the genetic modification of plants. All of these methods are based on introducing a foreign DNA into the plant cell, isolation of those cells containing the foreign DNA integrated into the genome, followed by subsequent regeneration of a whole plant. Unfortunately, such methods produce transformed cells that contain the introduced foreign DNA inserted randomly throughout the genome and often in multiple copies.

The random insertion of introduced DNA into the genome of host cells can be lethal if the foreign DNA happens to insert into, and thus mutate, a critically important native gene. In addition, even if a random insertion event does not impair the functioning of a host cell gene, the expression of an inserted foreign gene may be influenced by “position effects” caused by the surrounding genomic DNA. In some cases, the gene is inserted into sites where the position effects are strong enough to prevent the synthesis of an effective amount of product from the introduced gene. In other instances, overproduction of the gene product has deleterious effects on the cell.

Transgene expression is typically governed by the sequences, including promoters and enhancers, which are physically linked to the transgene. Currently, it is not possible to precisely modify the structure of transgenes once they have been introduced into plant cells. In many applications of transgene technology, it would be desirable to introduce the transgene in one form, and then be able to modify the transgene in a defined manner. By this means, transgenes could be activated or inactivated where the sequences that control transgene expression can be altered by either removing sequences present in the original transgene or by inserting additional sequences into the transgene.

For higher eukaryotes, homologous recombination is an essential event participating in processes like DNA repair and chromatid exchange during mitosis and meiosis. Recombination depends on two highly homologous extended sequences and several auxiliary proteins. Strand separation can occur at any point between the regions of homology, although particular sequences may influence efficiency. These processes can be exploited for a targeted integration of transgenes into the genome of certain cell types.

Even with the advances in genetic modification of higher plants, the major problems associated with the conventional gene transformation techniques have remained essentially unresolved as to the problems discussed above relating to variable expression levels due to chromosomal position effects and copy number variation of transferred genes. For these reasons, efficient methods are needed for targeting and control of insertion of nucleotide sequences to be integrated into a plant genome.

Transformation is now possible in many plant species. It has been used to introduce traits relatively rapidly when compared to introducing traits by conventional breeding methods. Most plant transformation protocols result in the transgene being integrated into the plant genome at a random location. The non-directed integration of the transgene can result in various problems. For instance, a mutation may be caused due to the location of integration. Another problem that occurs is variability in the transgene's expression due to its location of integration. This is called the “position effect”. Another disadvantage of non-directed integration occurs when an additional gene is transformed into an already transformed plant. Breeding in order to transfer two transgenes of interest is more cumbersome if the transgenes are located in different areas of the genome than if the transgenes are closely linked. Another problem that occurs in most transformation methods is the imperfect integration of the transgene. This imperfect integration, loss or rearrangement of nucleotides, can cause a change in expression level or total loss of gene function. Non-directed integration and imperfect integration necessitate a large number of transformation events to be made and screened before a desired transformation event is identified.

### SUMMARY OF THE INVENTION

The present invention relates to DNA integration wherein a recombinase system is used for integration and wherein at least two recombinase-mediated gene traps are produced.

### DETAILED DESCRIPTION OF THE INVENTION

A “recombinase” is any enzyme that catalyzes recombinase-mediated recombination between its corresponding recombination sites.

“Operably linked” means that the nucleotides are aligned so that they effectively function as a gene.

“Corresponding recombinase recognition sites” or “corresponding recombination sites” are at least two portions of DNA that can be cleaved and ligated together in the presence of a given recombinase. It is recognized that the recombinase, which can be used in the invention, will depend upon the recombination sites in the target site of the transformed plant and the targeting cassette. That is, if FRT sites are utilized, the FLP recombinase will be needed. In the same manner, where lox sites are utilized, the Cre recombinase is required. If the recombination sites comprise both a FRT and a lox site, both the FLP and Cre recombinase will be required in the plant cell.

“Non-identical recombination sites” are portions of DNA that will not recombine with each other.

“Corresponding recombinase” is any enzyme that catalyzes recombinase-mediated recombination between two recombinase recognition sites.

A “recombinase-mediated integration” or a “recombinase-mediated exchange” or a “recombinase-induced integration” is the exchange of DNA between two polynucleotides wherein the DNA located between two recombinase recognition sites located on the first polynucleotide is exchanged with the DNA located between two corresponding recombinase recognition sites on the second polynucleotide. The exchange of DNA happens in the presence of a recombinase. The mechanism of the exchange can vary (Guo, Feng et al. (1997) Nature 389:40-46 and Kosninsky et al. (2000) Plant J. 23:715-722).

A “recombinase-mediated gene trap” is a polynucleotide made from two polynucleotides that have been operably linked together by recombination or translocation.

A “recombinase-mediated gene trap element” is a polynucleotide region in proximity with at least one recombination recognition site in a target or donor locus which upon recombination-mediated exchange forms part of a recombinase mediated gene trap region. It is a polynucleotide that when operably linked together with a second polynucleotide by recombination or translocation has the potential to form a recombinase-mediated gene trap.

A “recombinase-mediated gene trap region” refers to a region of a polynucleotide sequence, either in a target or donor locus, resulting from a recombinase-mediated exchange and which includes at least two gene trap elements, one of which has been inserted through recombination from a polynucleotide in the other (target or donor) locus.

A “functional integration” or a “functional recombination” is an integration of DNA wherein the sequence has not been altered enough as to prevent transcription or as to prevent the expected gene product from being produced.

A “cassette” is a group of nucleotide sequences that lie in tandem. A cassette is usually integrated or exchanged as a unit. For example, a DNA cassette can be the DNA that is used in transformation. It can also be the DNA that gets integrated during recombinase-mediated integration.

“Fragments” and “variants” of the nucleotide sequences encoding recombinases and fragments and variant of recombinase proteins can also be used in the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence implements a recombination event. By “variants” is intended substantially similar sequences. For nucleotide sequences, conservative variants include those sequences that, because of the degeneracy of the genetic code, encode an amino acid sequence that retains the biological activity of a recombinase polypeptide.

As used herein “promoter” is a region of DNA to which an RNA molecule polymerase and other proteins bind to initiate transcription.

A “marker gene” is a sequence of DNA that when expressed allows it to be identified. A marker may be a selectable marker gene, a gene of interest or any gene that produces an identifiable product. The product is either screenable, scorable, visible or detectable. For example, any gene that produces a protein that can be detected through an ELISA may be considered a marker gene.

A “gene of interest” is any gene which, when transferred to a plant or plant cell, confers a desired characteristic. For example any gene that confers virus resistance, insect resistance, disease resistance, pest resistance, herbicide resistance, improved nutritional value, improved yield, change in fertility, production of a useful enzyme or metabolite in a plant could be a gene of interest.

A “polynucleotide of interest” is any DNA sequence that when transferred to a plant or plant cell, confers a desired characteristic. For example the polynucleotide of interest may be, but is not limited to, an anti-sense sequence, sequence fragment, a sequence that co-suppresses, micro-RNA, or a sequence that forms a hairpin. Other examples are any DNA sequence that confers virus resistance, insect resistance, disease resistance, pest resistance, herbicide resistance, improved nutritional value, improved yield, change in fertility, production of a useful enzyme or metabolite in a plant could be a polynucleotide of interest.

A “selectable marker” is any gene whose expression in a cell gives the cell a selective advantage. The selective advantage possessed by the cells with the selectable marker gene may be due to their ability to grow in the presence of a negative selective agent, such as a antibiotic or a herbicide, compared to the ability to grow cells not containing the gene. The selective advantage possessed by the cells containing the gene may also be due to their enhanced capacity to utilize an added compound such as a nutrient, growth factor or energy source.

As used herein a “sexual cross”, “cross” and “sexually crossing” encompass any means by which two haploid gametes are brought together resulting in a successful fertilization event and the production of a zygote. By “gamete” is intended a specialized haploid cell, either a sperm or an egg, serving for sexual reproduction. By “zygote” is intended a diploid cell produced by fusion of a male and female gamete (i.e. a fertilized egg). The resulting “hybrid” zygote contains chromosomes from both the acceptor and donor plant. The zygote then undergoes a series of mitotic divisions to form an embryo.

As defined herein, a “genetically modified plant cell” is a cell that comprises a stably integrated DNA sequence of interest.

As defined herein, the “transgenic plant” is a plant that comprises a stably integrated DNA sequence of interest.

DNA integration recombinase systems involve DNA cassettes one, which can be identified as “donor DNA” and one, which can be identified as “target DNA”. The target DNA generally comprises at least two recombinase recognition sites. The sites flank a polynucleotide that may comprise a gene or a set of gene expression cassettes. In the present invention the recombination recognition sites can be identical and/or non-identical. The donor DNA generally comprises at least two recombinase recognition sites. The sites flank a polynucleotide that may comprise a gene or a set of gene expression cassettes. DNA integration recombinase systems also have one or more proteins, called recombinases, which mediate the specific cleavage and ligation of the recombinase recognition sites. The recombinases can enter the system in various ways. For instance, a polynucleotide encoding the recombinase could be within the target DNA, the donor DNA, within the genome of a target plant, or within the genome of the donor plant. The recombinase could also enter the system via transient expression or as an active recombinase. The donor DNA can be initially integrated into the plant cell through transformation. After the donor DNA has been stably integrated into the plant cell, more genetically modified cells can be propagated from the transformed plant cell or plants can be obtained from the transformed plant cells and the donor DNA can be inherited via sexual and asexual reproduction. The target DNA can also be initially integrated into the plant cell through transformation. After the target DNA is stably integrated into the plant cell more genetically modified cells can be propagated from the transformed plant cell or plants can be obtained from the transformed plant then cells and the target DNA can be inherited via sexual and asexual reproduction.

After the donor DNA and the target DNA have been stably integrated into separate plants, creating a donor plant and a target plant, the plants then can be sexually crossed. Recombinase-mediated integration can occur with the crossing of the donor plant and the target plant in the presence of corresponding recombinase. The term “crossing” does not designate which plant is to be used as a male and which plant is to be used as a female, thus for purposes of this invention the plant containing the target DNA can be used as either the male or female in the cross.

The donor DNA and the target DNA can also be brought together through transformation of cells. If the donor DNA is stably integrated into a cell, the target DNA can then be used to transform the cell. In the presence of corresponding recombinase, recombinase-mediated integration can occur. If the target DNA is stably integrated into a cell, the donor DNA then can be used to transform the cell. Once again in the presence of corresponding recombinase, recombinase-mediated integration can occur.

The present invention provides a method for obtaining a genetically modified plant cell wherein a functional recombination at both the 5′ and 3′ ends of the polynucleotide can be identified. Said method comprising the steps of a) obtaining a plant cell that has a stably integrated first polynucleotide comprising at least two recombinase recognition sites and at least two recombinase-mediated gene trap elements; b) introducing into said plant cell a second polynucleotide comprising at least two recombinase recognition sites corresponding to the recombinase recognition sites of the first polynucleotide and at least two recombinase-mediated gene trap elements; c) having active recombinase present during said introduction; and d) identifying a plant cell comprising recombinase-mediated integration of the second polynucleotide at the chromosomal location of the first polynucleotide. The present invention allows one to screen for a sound recombination at both the 5′ and the 3′ ends of the cassette without the need for PCR or other time consuming molecular analysis.

In one embodiment of this invention a first polynucleotide comprising a promoter and a second polynucleotide comprising a coding sequence are linked through recombination or translocation. In another embodiment of the invention a coding sequence is divided at any point into a first and second polynucleotide, subsequently the two polynucleotides are operably linked through recombination or translocation. The opportunity for recombination or translocation of the two polynucleotides can be achieved through transformation or pollination.

In one embodiment, a polynucleotide in a target locus, upon recombination with a polynucleotide in a donor locus, comprises two recombinase-mediated gene trap regions, wherein each of the recombinase-mediated gene trap elements, one of which is provided from the polynucleotide in the donor locus, oriented upon recombination to comprise at least one regulatory sequence operably linked to a coding region of a gene of interest, where the coding region of the gene of interest is provided by one gene trap element, and the regulatory sequence is provided by the other gene trap element.

In another embodiment, a polynucleotide in a donor locus, upon recombination with a polynucleotide in a target locus, comprises two recombinase-mediated gene trap regions, wherein each of the recombinase-mediated gene trap regions comprises two gene trap elements, one of which is provided from the polynucleotide in the target locus, oriented upon recombination to comprise at least one regulatory sequence operably linked to a coding region of a gene of interest where the coding region of the gene of interest is provided by one gene trap element, and the regulatory sequence is provided by one gene trap element, and the regulatory sequence is provided by the other gene trap element.

Examples of recombination sites for use in the invention are known in the art and include FRT sites (See, for example, U.S. Pat. No. 6,187,994; Schlake and Bode (1994) Biochemistry 33:12746-12751; Huang et al. (1991) Nucleic Acids Research 19:443-448; Paul D. Sadowski (1995) In Progress in Nucleic Acid Research and Molecular Biology 51:53-91; Michael M. Cox (1989) In Mobile DNA, Berg and Howe (eds) American Society of Microbiology, Washington D.C., pp. 116-670; Dixon et al. (1995) 18:449-458; Umlauf and Cox (1988) The EMBO Journal 7:1845-1852; Buchholz et al. (1996) Nucleic Acids Research 24:3118-3119; Kilby et al. (1993) Trends Genet. 9:413-421; Rossant and Geagy (1995) Nat. Med. 1:592-594; Albert et al. (1995) The Plant J. 7:649-659; Bayley et al. (1992) Plant Mol. Biol. 18:353-361; Odell et al. (1990) Mol. Gen. Genet. 223:369-378; and Dale and Ow (1991) Proc. Natl. Acad. Sci. USA 88:10558-105620; all of which are herein incorporated by reference); lox (Albert et al. (1995) Plant J. 7:649-659; Qui et al. (1994) Proc. Natl. Acad. Sci. USA 91:1706-1710; Stuurman et al. (1996) Plant Mol. Biol. 32:901-913; Odell et al. (1990) Mol. Gen. Gevet. 223:369-378; Dale et al. (1990) Gene 91:79-85; and Bayley et al. (1992) Plant Mol. Biol. 18:353-361.) Dissimilar recombination sites are designed such that integrative recombination events are favored over the excision reaction. Such dissimilar recombination sites are known in the art. For example, Albert et al. introduced nucleotide changes into the left 13bp element (LE mutant lox site) or the right 13 bp element (RE mutant lox site) of the lox site. Recombination between the LE mutant lox site and the RE mutant lox site produces the wild-type loxP site and a LE+RE mutant site that is poorly recognized by the recombinase Cre, resulting in a stable integration event (Albert et al. (1995) Plant J. 7:649-659). See also, for example, Araki et al. (1997) Nucleic Acid Research 25:868-872.

Various recombinases can be used in this invention. For reviews of site-specific recombinases, see Sauer (1994) Current Opinion in Biotechnology 5:521-527; and Sadowski (1993) FASEB 7:760-767; the contents of which are incorporated herein by reference. The recombinase used in the methods of the invention can be a naturally occurring recombinase or an active fragment or variant of the recombinase. Recombinases useful in the methods and compositions of the invention include recombinases from the Integrase and Resolvase families, biologically active variants and fragments thereof, and any other naturally occurring or recombinantly produced enzyme or variant thereof, that catalyzes conservative site-specific recombination between specified DNA recombination sites. The Integrase family of recombinases has over one hundred members and includes, for example, FLP, Cre, Int and R. For other members of the Integrase family, see for example, Esposito et al. (1997) Nucleic Acid Research 25:3605-3614 and Abremski et al. (1992) Protein Engineering 5:87-91, both of which are herein incorporated by reference. Such recombination systems include, for example, the streptomycete bacteriophage phi C31 (Kuhstoss et al. (1991) J. Mol. Biol. 20:897-908); the SSV1 site-specific recombination system from Sulfolobus shibatae (Maskhelishvili et al. (1993) Mol. Gen. Genet. 237:334-342); and a retroviral integrase-based integration system (Tanaka et al. (1998) Gene 17:67-76). In other embodiments, the recombinase is one that does not require cofactors or a supercoiled substrate. Such recombinases include Cre, FLP, or active variants or fragments thereof. See U.S. Pat. No. 5,929,301.

The FLP recombinase is a protein that catalyzes a site-specific reaction that is involved in amplifying the copy number of the two-micron plasmid of S. cerevisiae during DNA replication. The FLP recombinase catalyzes site-specific recombination between two FRT sites. The FLP protein has been cloned and expressed. See, for example, Cox (1993) Proc. Natl. Acad. Sci. USA 80:4223-4227. The FLP recombinase for use in the invention may be that derived from the genus Saccharomyces. One can also synthesize the recombinase using plant-preferred codons for optimal expression in a plant of interest. A recombinant FLP enzyme encoding by a nucleotide sequence comprising maize preferred codons (moFLP) that catalyzes site-specific recombination events is known. See, for example, U.S. Pat. No. 5,929,301, herein incorporated by reference. Additional functional variants and fragments of FLP are known. See, for example, Hartung et al. (1998) J. Biol. Chem. 273:22884-22891 and Saxena et al. (1997) Biochim Biophys Acta 1340(2):187-204, and Hartley et al. (1980) Nature 286:860-864, all of which are herein incorporated by reference.

The bacteriophage recombinase Cre catalyzes site-specific recombination between two lox sites. The Cre recombinase is known in the art. See, for example, Guo et al. (1997) Nature 389:4046; Abremski et al. (1984) J. Biol. Chem. 259:1509-1514; Chen et al. (1996) Somat. Cell Mol. Genet. 22:477488; and Shaikh et al. (1977) J. Biol. Chem. 272:5695-5702, all of which are herein incorporated by reference. The Cre sequences may also be synthesized using plant-preferred codons. Such sequences (moCre) are described in WO 99/25840, herein incorporated by reference.

It is further recognized that chimeric recombinases can be used in the methods of the present invention. By “chimeric recombinase” is intended a recombinant fusion protein which is capable of catalyzing site-specific recombination between recombination sites that originate from different recombination systems. That is, if the non-identical recombination sites utilized in the present invention comprise FRT and loxP sites, a chimeric FLP/Cre recombinase or active variant thereof will be needed or both recombinases may be separately provided. Methods for the production and use of such chimeric recombinases or active variant thereof are described in U.S. Pat. No. 6,262,341 and U.S. Pat. No. 6,541,231, herein incorporated by reference.

There are many genes of interest or polynucleotides of interest that can be used as trangenes and therefore can be used in this invention. Exemplary transgenes implicated in this regard include, but are not limited to, those categorized below.

1. Transgenes that Confer Resistance to Pests or Disease and that Encode:

(A) Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae). A plant resistant to a disease is one that is more resistant to a pathogen as compared to the wild type plant.

(B) A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection (Rockville, Md.), for example, under ATCC Accession Nos. 40098, 67136, 31995 and 31998. Other examples of Bacillus thuringiensis transgenes being genetically engineered are given in the following patents and hereby are incorporated by reference for this purpose: U.S. Pat. Nos. 5,188,960; 5,689,052; 5,880,275; and WO 97/40162.

(C) A lectin. See, for example, the disclosure by Van Damme et al., Plant Molec. Biol. 24:25 (1994), who disclose the nucleotide sequences of several Clivia miniata mannose-binding lectin genes.

(D) A vitamin-binding protein such as avidin. See PCT application US93/06487 the contents of which are hereby incorporated by reference for this purpose. The application teaches the use of avidin and avidin homologues as larvicides against insect pests.

(E) An enzyme inhibitor, for example, a protease inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), and Sumitani et al., Biosci. Biotech. Biochem. 57:1243 (1993) (nucleotide sequence of Streptomyces nitrosporeus α-amylase inhibitor) and U.S. Pat. No. 5,494,813.

(F) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT Application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol.23:691 (1993), who teach the nucleotide sequence of a cDNA encoding tobacco hookworm chitinase, and Kawalleck et al., Plant Molec. Biol. 21:673 (1993), who provide the nucleotide sequence of the parsley ubi4-2 polyubiquitin gene.

(G) A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol.104:1467 (1994), who provide the nucleotide sequence of a maize calmodulin cDNA clone.

(H) A hydrophobic moment peptide. See PCT Application WO95/16776 (disclosure of peptide derivatives of Tachyplesin which inhibit fungal plant pathogens) and PCT Application WO95/18855 (teaches synthetic antimicrobial peptides that confer disease resistance), the respective contents of which are hereby incorporated by reference for this purpose.

(I) A membrane permease, a channel former or a channel blocker. For example, see the disclosure by Jaynes et al., Plant Sci. 89:43 (1993), of heterologous expression of a cecropin-β lytic peptide analog to render transgenic tobacco plants resistant to Pseudomonas solanacearum.

(J) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol.28:451 (1990). Coat protein-induced resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus.

(K) A developmental-arrestive protein produced in nature by a pathogen or a parasite. Thus, fungal endo α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

(L) A developmental-arrestive protein produced in nature by a plant. For example, Logemann et al., Bio/Technology 10:305 (1992), have shown that transgenic plants expressing the barley ribosome-inactivating gene have an increased resistance to fungal disease.

(M) Genes involved in the Systemic Acquired Resistance (SAR) Response and/or the pathogenesis related genes. Briggs, S., Current Biology 5:128-131 (1995).

(N) Antifungal genes (Cornelissen and Melchers, PI. Physiol. 101:709-712, (1993) and Parijs et al., Planta 183:258-264, (1991) and Bushnell et al., Can. J. of Plant Path. 20(2):137-149 (1998).

2. Transgenes that Confer Resistance to an Herbicide, for Example:

(A) A herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively. See also, U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270, which are incorporated herein by reference for this purpose.

(B) Glyphosate which has resistance imparted by mutant 5-enolpyruvl-3-phosphikimate synthase (EPSP) and aroA genes, respectively. See, for example, U.S. Pat. No. 4,940,835 to Shah et al., which discloses the nucleotide sequence of a form of EPSPS which can confer glyphosate resistance. U.S. Pat. No. 5,627,061 to Barry et al. also describes genes encoding EPSPS enzymes. See also U.S. Pat. Nos. 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E; and 5,491,288; and international publications WO 97/04103; WO 97/04114; WO 00/66746; WO 01/66704; WO 00/66747 and WO 00/66748, which are incorporated herein by reference for this purpose. Glyphosate resistance is also imparted to plants that express a gene that encodes a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase (GAT). See, for example, PCT publication WO02/36782 and U.S. application Ser. No. 10/427,692. A DNA molecule encoding a mutant aroA gene can be obtained under ATCC Accession No. 39256, and the nucleotide sequence of the mutant gene is disclosed in U.S. Pat. No. 4,769,061 to Comai.

(C). Phosphono compounds such as glufosinate (phosphinothricin acetyl transferase (PAT) and Streptomyces hygroscopicus phosphinothricin acetyl transferase (bar) genes. The nucleotide sequence of a phosphinothricin-acetyl-transferase gene is provided in European Patent No. 0 242 246 and 0 242 236 to Leemans et al. De Greef et al., Bio/Technology 7:61 (1989), describe the production of transgenic plants that express chimeric bar genes coding for phosphinothricin acetyl transferase activity. European patent application No. 0 333 033 to Kumada et al. and U.S. Pat. No. 4,975,374 to Goodman et al. disclose nucleotide sequences of glutamine synthetase genes which confer resistance to herbicides such as L-phosphinothricin. See also, U.S. Pat. Nos. 5,969,213; 5,489,520; 5,550,318; 5,874,265; 5,919,675; 5,561,236; 5,648,477; 5,646,024; 6,177,616 B1; and 5,879,903, which are incorporated herein by reference for this purpose.

(D) Pyridinoxy or phenoxy proprionic acids and cycloshexones (ACCase inhibitor-encoding genes). Exemplary of genes conferring resistance to phenoxy proprionic acids and cycloshexones, such as sethoxydim and haloxyfop, are the Acc1-S1, Acc1-S2 and Acc1-S3 genes described by Marshall et al., Theor. Appl. Genet. 83:435 (1992).

(E) A herbicide that inhibits photosynthesis, such as a triazine (psbA and gs+ genes) and a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285:173 (1992).

(F) Acetohydroxy acid synthase, which has been found to make plants that express this enzyme resistant to multiple types of herbicides, has been introduced into a variety of plants (see, e.g., Hattori et al. (1995) Mol Gen Genet 246:419). Other genes that confer tolerance to herbicides include: a gene encoding a chimeric protein of rat cytochrome P4507A1 and yeast NADPH-cytochrome P450 oxidoreductase (Shiota et al. (1994) Plant Physiol 106:17), genes for glutathione reductase and superoxide dismutase (Aono et al. (1995) Plant Cell Physiol 36:1687, and genes for various phosphotransferases (Datta et al. (1992) Plant Mol Biol 20:619).

(G) Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306 B1; 6,282,837 B1; and 5,767,373; and international publication WO 01/12825, which are incorporated herein by reference for this purpose.

3. Transgenes that Confer or Contribute to a Grain Trait, Such as:

(A) Modified fatty acid metabolism, for example, by transforming a plant with a gene that suppresses stearoyl-ACP desaturase to increase stearic acid content of the plant. See Knultzon et al., Proc. Natl. Acad. Sci. USA 89:2624 (1992).

(B) Phytate content

(1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene.

(2) A gene could be introduced that reduces phytate content. Examples of genes are disclosed in U.S. Pat. Nos. 6,197,561; 6,291,224 and WO 02/059324.

(C) Modified carbohydrate composition effected, for example, by transforming plants with a gene coding for an enzyme that alters the branching pattern of starch. See Shiroza et al., J. Bacteriol. 170:810 (1988) (nucleotide sequence of Streptococcus mutans fructosyltransferase gene), Steinmetz et al., Mol. Gen. Genet. 200:220 (1985) (nucleotide sequence of Bacillus subtilis levansucrase gene), Pen et al., Bio/Technology 10:292 (1992) (production of transgenic plants that express Bacillus licheniformis α-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley α-amylase gene), and Fisher et al., Plant Physiol. 102:1045 (1993) (maize endosperm starch branching enzyme II). U.S. Pat. Nos. 6,43,886 and 6,399,859 disclose starch synthase genes in maize.

(D) Elevated oleic acid via FAD-2 gene modification and/or decreased linolenic acid via FAD-3 gene modification (see U.S. Pat. Nos. 6,063,947; 6,323,392; and WO 93/11245).

4. Genes that Control Male-Sterility

(A) Introduction of a deacetylase gene under the control of a tapetum-specific promoter and with the application of the chemical N-Ac-PPT (WO 01/29237).

(B) Introduction of various stamen-specific promoters (WO 92/13956, WO 92/13957).

(C) Introduction of the bamase and the barstar gene (Paul et al., Plant Mol. Biol. 19:611-622, 1992).

There are also many promoters that can be used as in this invention. Exemplary promoters implicated in this regard include, but are not limited to, the following. “Constitutive” promoters are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter, and other transcription initiation regions from various plant genes known to those of skill.

Alternatively, a promoter can direct expression of a polynucleotide of interest in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds, or flowers. Exemplary promoters include the root cdc2a promoter (Doerner, P., et al. (1996) Nature 380:520-523) or the root peroxidase promoter from wheat (Hertig, C., et al. (1991) Plant Mol. Biol. 16:171-174).

Both heterologous and non-heterologous (i.e., endogenous) promoters can be employed to direct expression of the polynucleotide of interest.

Isolated nucleic acids which serve as promoter or enhancer elements can be introduced in the appropriate position (generally upstream) of a non-heterologous form of a polynucleotide of the present invention so as to up- or down-regulate expression of a polynucleotide of the present invention. For example, endogenous promoters can be altered in vivo by mutation, deletion, and/or substitution (see, Kmiec, U.S. Pat. No. 5,565,350; Zarling et al., PCT/US93/03868), or isolated promoters can be introduced into a plant cell in the proper orientation and distance from a gene of the present invention so as to control the expression of the gene. Gene expression can be modulated under conditions suitable for plant growth so as to alter the total concentration and/or alter the composition of the polypeptides of the present invention in plant cell.

The DNA cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods or sequences known to enhance translation can also be utilized, for example, introns, and the like.

In preparing a DNA cassette, various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The method of transformation is not critical to the invention; various methods of transformation are currently available. As newer methods are available to transform host cells they may be directly applied. Accordingly, a wide variety of methods have been developed to insert a DNA sequence into the genome of a host cell to obtain the transcription and/or translation of the sequence. Thus, any method that provides for efficient transformation/transfection may be employed.

Methods for transforming various host cells are disclosed in Klein et al. “Transformation of microbes, plants and animals by particle bombardment”, Bio/Technol. New York, N.Y., Nature Publishing Company, March 1992, 10(3):286-291. Techniques for transforming a wide variety of higher plant species are well known and described in the technical, scientific, and patent literature. See, for example, Weising et al., Ann. Rev. Genet. 22:421477 (1988).

For example, the DNA construct may be introduced directly into the genomic DNA of the plant cell using techniques such as electroporation, PEG-induced transfection, particle bombardment, silicon fiber delivery, or microinjection of plant cell protoplasts or embryogenic callus. See, e.g., Tomes et al., Direct DNA Transfer into Intact Plant Cells Via Microprojectile Bombardment. pp.197-213 in Plant Cell, Tissue and Organ Culture, Fundamental Methods. eds. O. L. Gamborg and G. C. Phillips. Springer-Verlag Berlin Heidelberg New York, 1995. The introduction of DNA constructs using polyethylene glycol precipitation is described in Paszkowski et al., Embo J. 3:2717-2722 (1984). Electroporation techniques are described in Fromm et al., Proc. Natl. Acad. Sci. 82:5824 (1985). Ballistic transformation techniques are described in Klein et al., Nature 327:70-73 (1987).

Alternatively, the DNA constructs may be combined with suitable T-DNA flanking regions and introduced into a Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-meditated transformation techniques are well described in the scientific literature. See, for example Horsch et al., Science 233:496-498 (1984), and Fraley et al., Proc. Natl. Acad. Sci. 80:4803 (1983). For instance, Agrobacterium transformation of maize is described in U.S. Pat. No. 5,981,840. Agrobacterium transformation of monocot is found in U.S. Pat. No. 5,591,616. Agrobacterium transformation of soybeans is described in U.S. Pat. No. 5,563,055.

Other methods of transformation include (1) Agrobacterium rhizogenes-induced transformation (see, e.g., Lichtenstein and Fuller In: Genetic Engineering, vol. 6, PWJ Rigby, Ed., London, Academic Press, 1987; and Lichtenstein, C. P., and Draper, J,. In: DNA Cloning, Vol. II, D. M. Glover, Ed., Oxford, IRI Press, 1985), Application PCT/US87/02512 (WO 88/02405 published Apr. 7, 1988) describes the use of A. rhizogenes strain A4 and its Ri plasmid along with A. tumefaciens vectors pARC8 or pARC16 (2) liposome-induced DNA uptake (see, e.g., Freeman et al., Plant Cell Physiol. 25:1353, 1984), (3) the vortexing method (see, e.g., Kindle, Proc. Natl. Acad. Sci., USA 87:1228, (1990).

DNA can also be introduced into plants by direct DNA transfer into pollen as described by Zhou et al., Methods in Enzymology 101:433 (1983); D. Hess, Intern Rev. Cytol. 107:367 (1987); Luo et al., Plant Mol. Biol. Reporter, 6:165 (1988). Expression of polypeptide coding nucleic acids can be obtained by injection of the DNA into reproductive organs of a plant as described by Pena et al., Nature 325:274 (1987). Transformation can also be achieved through electroporation of foreign DNA into sperm cells then microinjecting the transformed sperm cells into isolated embryo sacs as described in U.S. Pat. No. 6,300,543 by Cass et al. DNA can also be injected directly into the cells of immature embryos and the rehydration of desiccated embryos as described by Neuhaus et al., Theor. Appl. Genet. 75:30 (1987); and Benbrook et al., in Proceedings Bio Expo 1986, Butterworth, Stoneham, Mass., pp. 27-54 (1986).

Transformed plant cells which are derived by any of the above transformation techniques can be cultured to regenerate a whole plant which possesses the transformed genotype. Such regeneration techniques often rely on manipulation of certain phytohormones in a tissue culture growth medium, typically relying on a biocide and/or herbicide marker which has been introduced together with a polynucleotide of the present invention. For transformation and regeneration of maize see, Gordon-Kamm et al., The Plant Cell 2:603-618 (1990).

The following examples are offered by way of illustration and not by way of limitation.

Description of Polynucleotide Elements Used in the Examples

amCyan—encodes a variant of wild-type Anemonia majano cyan fluorescent protein that has been engineered for brighter fluorescence.

Actin Pro-rice actin promoter. See McElroy et al. (1990) Plant Cell 2:163-171.

CaMV35S Pro—indicates the promoter sequence from the Cauliflower Mosiac Virus gene. See Odell et al. (1985) Nature 313:810-812.

CaMV35S Term—indicates the termination sequence from the Cauliflower Mosiac Virus gene. See Odell et al. (1985) Nature 313:810-812.

crc-See Bruce et al. (2000) Plant Cell 12(1):65-79.

cre—indicates the polynucleotide encoding Cre recombinase. The bacteriophage recombinase Cre catalyzes site-specific recombination between two lox sites. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477488; and Shaikh et al. (1977) J. Biol. Chem. 272:5695-5702. All of which are herein incorporated by reference. Such Cre sequence may also be synthesized using plant preferred codons.

bar—The expression of bar confers resistance to bialaphos. See Thompson et al. EMBO J. 9(1987) 2519-2523.

FRT1—indicates the wild type recombination sequence. See U.S. Pat. No. 6,187,994.

FRT5—indicates a recombination sequence recognized by the FLP recombinase. See U.S. Pat. No. 6,187,994.

FRT6—indicates a recombination sequence recognized by the FLP recombinase. See U.S. Pat. No. 6,187,994.

FRT7—indicates a recombination sequence recognized by the FLP recombinase. See U.S. Pat. No. 6,187,994.

FLP-indicates the DNA sequence encoding FLP recombinase. FLP recombinase is a protein which catalyzes a site-specific reaction that is involved in amplifying the copy number of the two micron plasmid of S. cerevisiae during DNA replication. FLP protein has been cloned and expressed. See, for example, Cox (1993) Proc. Natl. Acad. Sci. U.S.A. 80:4223-4227. The FLP recombinase for use in the invention may be that derived from the genus Saccharomyces. It may be preferable to synthesize the recombinase using plant preferred codons for optimum expression in a plant of interest. See, for example, U.S. Pat. No. 5,929,301, entitled Novel Nucleic Acid Sequence Encoding FLP Recombinase, herein incorporated by reference.

gat—genes encoding glyphosate N-acetyltransferase (GAT). See PCT publication WO02/36782 and U.S. application Ser. No. 10/427,692.

GLB1 Pro-indicates a maize globulin promoter. See Liu, S., et al. (1996) Plant Cell Reports 16:158-162.

gm-als-indicates a soybean als gene. See publication WO0037662. gus-indicates the Beta-glucuronidase (GUS) sequence (Jefferson et al. (1991) In Plant Molecular Biology Manual (Gelvin et al., eds.), pp.1-33, Kluwer Academic Publishers).

hyg-encodes hygromycin resistance. See Van den Elzen et al. (1985) Plant Molecular Biology 5(5):299-302.

kti3-indicates a soybean Kunitz trysin inhibitor 3 gene. See Jofuku et al. 1989 Plant Cell 1:427435 and U.S. Pat. No. 6,459,019.

lec1—indicates a leafy cotyledon 1 transcriptional activator polynucleotide. See U.S. patent application Ser. No. 09/435,054.

LB—indicates left border.

lpt2 Pro-indicates a promoter form barely lipid transfer protein. See Kalla et al. Plant J. 6(6). 849-60.

moCah—is a maize optimized gene that encodes for the Myrothecium verrucaria cyanamide hydratase protein [CAH] that can hydrate cyanamide to non-toxic urea.

pinII—indicates potato proteinase inhibitor. See Ryan (1990) Ann. Rev. Phytopath. 28:425-449; Duan et al. (1996) Nature Biotechnology 14:494-498.

Pro—indicates a promoter sequence.

RB—indicates right border.

SCP1 Pro—indicates a promoter. See U.S. Pat. No. 6,072,050 Term-indicates a terminator sequence.

Ubi Pro—indicates a ubiquitin promoter. See Christensen et al. (1989) Plant Mol. Biol. 12:619-632 and Christensen et al. (1992) Plant Mol. Biol. 18:675-689) Ubi1ZM Pro-indicates a ubiquitin maize promoter.

YFP—indicates a polynucleotide that encodes yellow fluorescent protein. See U.S. Pat. No. 6,608,189.

DNA Delivery Methods

Maize

Transformation of the target or donor plasmids into maize follows a well-established bombardment transformation protocol used for introducing DNA into the scutellum of immature maize embryos (See, e.g., Tomes et al., Direct DNA Transfer into Intact Plant Cells Via Microprojectile Bombardment. pp.197-213 in Plant Cell, Tissue and Organ Culture, Fundamental Methods. eds. O. L. Gamborg and G. C. Phillips. Springer-Verlag Berlin Heidelberg New York, 1995.). It is noted that any suitable method of transformation can be used, such as Agrobacterium-mediated transformation and many other methods. Cells were transformed by culturing maize immature embryos (approximately 1-1.5 mm in length) onto medium containing N6 salts, Erikkson's vitamins, 0.69 g/l proline, 2 mg/I 2,4-D and 3% sucrose. After 4-5 days of incubation in the dark at 28° C., embryos were removed from the first medium and cultured onto similar medium containing 12% sucrose. Embryos were allowed to acclimate to this medium for 3 h prior to transformation. The scutellar surface of the immature embryos was targeted using particle bombardment. Embryos were transformed using the PDS-1000 Helium Gun from Bio-Rad at one shot per sample using 650PSI rupture disks. DNA delivered per shot averaged at 0.1667 μg. Following bombardment, all embryos were maintained on standard maize culture medium (N6 salts, Erikkson's vitamins, 0.69 g/l proline, 2 mg/I 2,4-D, 3% sucrose) for 2-3 days and then transferred to N6-based medium containing a selective agent. Plates were maintained at 28° C. in the dark and were observed for colony recovery with transfers to fresh medium every two to three weeks. Recovered colonies and plants are scored based on the selectable or screenable phenotype imparted by the marker gene(s) introduced (i.e. herbicide resistance, fluorescence or anthocyanin production), and by molecular characterization via PCR and Southern analysis.

Transformation of the target or donor DNA into Pioneer Hi-Bred International, Inc. proprietary maize inbreds PHN46 and PHP38 was done using the Agrobacterium mediated DNA delivery method, as described by U.S. Pat. No 5,981,840 with the following modifications. It is noted that any suitable method of transformation can be used, such as particle-mediated transformation, as well as many other methods. Agrobacteria were grown to the log phase in liquid minimal A medium containing 100 μM spectinomycin. Embryos were immersed in a log phase suspension of Agrobacteria adjusted to obtain an effective concentration of 5×10⁸ cfu/ml. Embryos were infected for 5 minutes and then co-cultured on culture medium containing acetosyringone for 7 days at 20° C. in the dark. After 7 days, the embryos were transferred to standard culture medium (MS salts with N6 macronutrients, 1 mg/L 2,4-D, 1 mg/L Dicamba, 20 g/L sucrose, 0.6 g/L glucose, 1 mg/L silver nitrate, and 100 mg/L carbenicillin) with a selective agent. Plates were maintained at 28° C. in the dark and were observed for colony recovery with transfers to fresh medium every two to three weeks. Recovered colonies and plants are scored based on the selectable or screenable phenotype imparted by the marker gene(s) introduced (i.e. herbicide resistance, fluorescence or anthocyanin production), and by molecular characterization via PCR and Southern analysis.

Soybean

Transformation of the target or donor polynucleotides can be accomplished through numerous well-established methods for plant cells, including for example particle bombardment, sonication, PEG treatment or electroporation of protoplasts, electroporation of intact tissue, silica-fiber methods, microinjection or Agrobacterium-mediated transformation. Using one of the above methods, DNA is introduced into soybean cells capable of growth on suitable soybean culture medium. The target or donor DNA is cloned into a cassette. Particle bombardment is used to introduce the cassette-containing plasmid into soybean cells capable of growth on suitable soybean culture medium containing a selective agent. Such competent cells can be from soybean suspension culture, cell culture on solid medium, freshly isolated cotyledonary nodes or meristem cells. Suspension-cultured somatic embryos of Jack, a Glycine max (I.) Merrill cultivar, are used as the target for the plasmid. Media for induction of cell cultures with high somatic embryogenic morphology, for establishing suspensions, and for maintenance and regeneration of somatic embryos are described (Bailey M A, Boerma H R, Parrott W A, 1993 Genotype effects on proliferative embryogenesis and plant regeneration of soybean, In Vitro Cell Dev Biol 29P:102-108). Likewise, methods for particle-mediated transformation of soybean are well established in the literature, see for example Stewart N C, Adang M J, All J N, Boerma H R, Cardineau G, Tucker D, Parrott W A, 1996, Genetic transformation, recovery and characterization of fertile soybean transgenic for a synthetic Bacillus thuringiensis crylAc gene, Plant Physiol 112:121-129.

Maintenance of Soybean Embryogenic Suspension Cultures

Soybean embryogenic suspension cultures are maintained in 35 ml liquid media SB196 or SB172 in 250 ml Erlenmeyer flasks on a rotary shaker, 150 rpm, 26 C with cool white fluorescent lights on 16:8 hr day/night photoperiod at light intensity of 30-35 uE/m2s.

Cultures are subcultured every two weeks by inoculating approximately 35 mg of tissue into 35 ml of fresh liquid media. Alternatively, cultures are initiated and maintained in 6-well Costar plates.

SB 172 media is prepared as follows: (per liter), 1 bottle Murashige and Skoog Medium (Duchefa #M 0240), 1 ml B5 vitamins 1000× stock, 1 ml 2,4-D stock (Gibco 11215-019), 60 g sucrose, 2 g MES, 0.667 g L-Asparagine anhydrous (GibcoBRL 11013-026), pH 5.7

SB 196 media is prepared as follows: (per liter) 10 ml MS FeEDTA, 10 ml MS Sulfate, 10 ml FN-Lite Halides, 10 ml FN-Lite P,B,Mo, 1 ml B5 vitamins 1000× stock, 1 ml 2,4-D, (Gibco 11215-019), 2.83 g KNO₃, 0.463 g (NH₄)₂SO₄, 2 g MES, 1 g Asparagine Anhydrous, Powder (Gibco 11013-026), 10 g Sucrose, pH 5.8.

2,4-D stock concentration 10 mg/ml is prepared as follows: 2,4-D is solubilized in 0.1 N NaOH, filter-sterilized, and stored at −20° C.

B5 vitamins 1000× stock is prepared as follows: (per 100 ml)—store aliquots at −20° C., 10 g myo-inositol, 100 mg nicotinic acid, 100 mg pyridoxine HCl, 1 g thiamine.

Particle Bombardment

Soybean embryogenic suspension cultures are transformed with various plasmids by the method of particle gun bombardment (Klein et al., 1987; Nature 327:70).

To prepare tissue for bombardment, approximately two flasks of suspension culture tissue that has had approximately 1 to 2 weeks to recover since its most recent subculture is placed in a sterile 60×20 mm petri dish containing 1 sterile filter paper in the bottom to help absorb moisture. Tissue (i.e. suspension clusters approximately 3-5 mm in size) is spread evenly across each petri plate. Residual liquid is removed from the tissue with a pipette, or allowed to evaporate to remove excess moisture prior to bombardment. Per experiment, 4-6 plates of tissue are bombarded. Each plate is made from two flasks.

To prepare gold particles for bombardment, 30 mg gold is washed in ethanol, centrifuged and resuspended in 0.5 ml of sterile water. For each plasmid combination (treatments) to be used for bombardment, a separate micro-centrifuge tube is prepared, starting with 50 μl of the gold particles prepared above. Into each tube, the following are also added; 5 μl of plasmid DNA (at 1 μg/μl), 50 μl CaCl₂, and 20 μl 0.1 M spermidine. This mixture is agitated on a vortex shaker for 3 minutes, and then centrifuged using a microcentrifuge set at 14,000 RPM for 10 seconds. The supernatant is decanted and the gold particles with attached, precipitated DNA are washed twice with 400 μl aliquots of ethanol (with a brief centrifugation as above between each washing). The final volume of 100% ethanol per each tube is adjusted to 40 ul, and this particle/DNA suspension is kept on ice until being used for bombardment.

Immediately before applying the particle/DNA suspension, the tube is briefly dipped into a sonicator bath to disperse the particles, and then 5 μg of DNA prep is pipetted onto each macro-carrier and allowed to dry. The macro-carrier is then placed into the DuPontS Biolistics PDS1000/HE gun. Using the DuPont® Biolistic PDS1000/HE instrument for particle-mediated DNA delivery into soybean suspension clusters, the following settings are used. The membrane rupture pressure is 1100 psi. The chamber is evacuated to a vacuum of 27-28 inches of mercury. The tissue is placed approximately 3.5 inches from the retaining/stopping screen (3rd shelf from the bottom). Each plate is bombarded twice, and the tissue clusters are rearranged using a sterile spatula between shots.

Following bombardment, the tissue is re-suspended in liquid culture medium, each plate being divided between 2 flasks with fresh SB196 or SB172 media and cultured as described above. Four to seven days post-bombardment, the medium is replaced with fresh medium containing a selection agent. The selection media is refreshed weekly for 4 weeks and once again at 6 weeks. Weekly replacement after 4 weeks may be necessary if cell density and media turbidity is high.

Four to eight weeks post-bombardment, green, transformed tissue may be observed growing from untransformed, necrotic embryogenic clusters. Isolated, green tissue is removed and inoculated into 6-well microtiter plates with liquid medium to generate clonally-propagated, transformed embryogenic suspension cultures.

Each embryogenic cluster is placed into one well of a Costar 6-well plate with 5 mls fresh SB196 media with the selective agent. Cultures are maintained for 2-6 weeks with fresh media changes every 2 weeks. When enough tissue is available, a portion of surviving transformed clones are subcultured to a second 6-well plate as a back-up to protect against contamination.

Regeneration of Soybean Somatic Embryos

To promote in vitro maturation, transformed embryogenic clusters are removed from liquid SB196 and placed on solid agar media, SB 166, for 2 weeks. Tissue clumps of 2-4 mm size are plated at a tissue density of 10 to 15 clusters per plate. Plates are incubated in diffuse, low light (<10 μE) at 26±1° C. After two weeks, clusters are subcultured to SB 103 media for 3-4 weeks.

SB 166 is prepared as follows: (per liter), 1 pkg. MS salts (Gibco/BRL—Cat#11117-017), 1 ml B5 vitamins 1000× stock, 60 g maltose, 750 mg MgCl2 hexahydrate, 5 g activated charcoal, pH 5.7, 2 9 gelrite.

SB 103 media is prepared as follows: (per liter), 1 pkg. MS salts (Gibco/BRL—Cat#11117-017), 1 ml B5 vitamins 1000× stock, 60 g maltose, 750 mg MgCl2 hexahydrate, pH 5.7, 2 g gelrite.

After 5-6 week maturation, individual embryos are desiccated by placing embryos into a 100×15 petri dish with a 1 cm2 portion of the SB103 media to create a chamber with enough humidity to promote partial desiccation, but not death.

Approximately 25 embryos are desiccated per plate. Plates are sealed with several layers of parafilm and again are placed in a lower light condition. The duration of the desiccation step is best determined empirically, and depends on size and quantity of embryos placed per plate. For example, small embryos or few embryos/plate require a shorter drying period, while large embryos or many embryos/plate require a longer drying period. It is best to check on the embryos after about 3 days, but proper desiccation will most likely take 5 to 7 days. Embryos will decrease in size during this process.

Desiccated embryos are planted in SB 71-1 or MSO medium where they are left to germinate under the same culture conditions described for the suspension cultures. When the plantlets have two fully-expanded trifoliolate leaves, germinated and rooted embryos are transferred to sterile soil and watered with a half-strength MS-salt solution. Plants are grown to maturity for seed collection and analysis. Embryogenic cultures from the SR treatment are expected to regenerate easily. Healthy, fertile transgenic plants are grown in the greenhouse. Seed-set on SR transgenic plants is expected to be similar to control plants, and transgenic progeny are recovered.

SB 71-1 is prepared as follows: 1 bottle Gamborg's B5 salts w/sucrose (Gibco/BRL—Cat#21153-036), 10 g sucrose, 750 mg MgCI2 hexahydrate, pH 5.7, 2 g gelrite.

MSO media is prepared as follows: 1 pkg Murashige and Skoog salts (Gibco 11117-066), 1 ml B5 vitamins 1000× stock, 30 g sucrose, pH 5.8, 2 g Gelrite.

### EXAMPLE 1

### Recombinase-Mediated Cassette Exchange Results in Activation of Two Marker Genes in the Target Locus

Inbred PHN46 is transformed using Agrobacterium, introducing the following “Target DNA” (arrows indicate direction of promoters near the FRT sites):

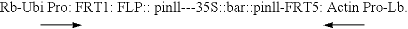

After the recovery period on non-selective medium, calli are selected on bialaphos-containing medium and regenerated to produce “Target” plants. The expression of bar confers resistance to bialaphos. DNA is extracted from regenerated T0 plants and subsequent T1 progeny to confirm that the above-introduced DNA is present as a single copy using standard Southern analysis methods (see Maniatus). The phenotype imparted by the above DNA elements to the “Target” plants is FLP recombinase activity and bialaphos resistance (FLP⁺, BLP^{r}).

In a separate transformation experiment, inbred PHN46 is transformed using Agrobacterium, introducing the following “Donor DNA”:
Rb-35S Pro::bar::pinII-FRT1:GAT::pinII—pinII::moCah:FRT5-Lb

After the recovery period on non-selective medium, calli are selected on bialaphos-containing medium and regenerated to produce “Donor” plants, with the phenotype of bialaphos resistance (BLP^{r}). The GAT and moCAH sequences have no promoters and thus are not expressed in the donor plants.

Target and donor plants are grown and upon reaching maturity are crossed to each other. In this scenario, the cassette from the donor containing inactive GAT and moCah sequences is removed from the donor locus and inserted into the target locus, in the process positioning the GAT sequence behind the Ubiquitin promoter (Ubi Pro) and the moCah sequence behind the Actin promoter (Actin Pro). The resultant functional orientation of these two structural sequences relative to the promoters results in expression of GAT and moCah and confers resistance to the herbicides Glyphosate (GLY^{r}) and Cyanimide (CYA^{r}), respectively. Thus, progeny seed from the above “Target×Donor” are planted and the resultant seedlings are sprayed with both herbicides. Progeny in which proper recombinase-mediated cassette exchange has occurred (recombination at both the FRT1 and FRT5 sites) are readily identified (phenotype FLP⁻, BLP^{r}, GLY^{r} and CYA^{r}). The two new herbicide resistance traits (GLY^{r} and CYA^{r}) that resulted from the cassette exchange in the target locus will continue to co-segregate along with any other DNA elements originally introduced into the target locus adjacent but outside the FRT sites (i.e. they behave as a linkage group), and the inactivated FLP along with the two copies of 35S::bar::pinII that are now located at the donor locus will also segregate as a single unit. Thus, these two loci can easily be segregated away from each other in the next generation, and Glyphosate/Cyanamide double-resistant plants are obtained. PCR analysis across the recombined FRT1 and FRT5 junctions, as well as Southern analysis and sequencing will be used to confirm that precise recombination mediated by FLP recombinase occurred during the cassette exchange.

### EXAMPLE 2

### Recombinase-Mediated Cassette Exchange Results in Activation of Two Marker Genes in the Donor Locus

Inbred PHN46 is transformed using Agrobacterium, introducing the following “Target DNA”:

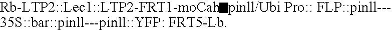

After the recovery period on non-selective medium, calli are selected on bialaphos-containing medium and regenerated to produce “Target” plants. DNA is extracted from regenerated T0 plants and subsequent T1 progeny to confirm that the above-introduced DNA is present as a single copy using standard Southern analysis methods (see Maniatus).

In a separate transformation experiment, inbred PHN46 is transformed using Agrobacterium, introducing the following “Donor DNA”:

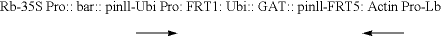

After the recovery period on non-selective medium, calli are selected on bialaphos-containing medium and regenerated to produce “Donor” plants.

Target and donor plants are grown and upon reaching maturity are crossed to each other. In this scenario, the cassette from the donor contains an active Ubi::GAT:pinII which upon successful recombinase-mediated cassette exchange will be inserted into the target locus which already contains a “high oil” trait conferred by LTP2::Lec1::LTP2 outside the FRT sites. In the F1 progeny, the desired product (linked GAT and Lec1 traits) cannot be discerned directly based on altered GAT or Lec1 phenotypes (neither the GAT sequence nor the Lec1 sequence were newly activated when the exchange occurred). However, in the process of cassette exchange, two inactive sequences originally in the target locus (moCah and YFP) are now juxtaposed with promoters in the donor locus and this double gene activation can be screened for as an indication that cassette exchange occurred. Progeny resistant to Cyanamide spraying that also exhibit yellow fluorescence in the leaves (as measured by a hand-held OS1-FL fluorescence meter; Opti-Sciences, Inc., 164 Westford Rd., Tyngsboro, Mass. 01879) show the proper phenotypes that indicate cassette exchange between the two loci. These CYA^{r}, YFP+ plants are grown and outcrossed to wild-type PHN46, and in the resultant progeny the CYA^{r}, YFP+ traits segregate as a single unit (now in the donor locus) and the GLY^{r}, LEC1 traits are now linked and segregate away from CYA^{r}, YFP+. PCR analysis across the recombined FRT1 and FRT5 junctions, as well as Southern analysis and sequencing will be used to confirm that precise recombination mediated by FLP recombinase occurred during the cassette exchange.

### EXAMPLE 3

### Recombinase-Mediated Cassette Exchange Using Two Independent Recombination Systems

Inbred PHN46 is transformed using Agrobacterium, introducing the following “Target DNA” (arrows indicate direction of promoters near the recombination sites):

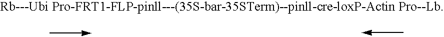

In this configuration, two required recombination events are independent from each other. The combined action of FLP and Cre on their respective recombination sites provides optimal environment for two recombination events that are required for the recombinase-mediated cassette exchange to take place.

After the recovery period on non-selective medium, calli are selected on bialaphos-containing medium and regenerated to produce “Target” plants. The expression of bar confers resistance to bialaphos. DNA is extracted from regenerated T0 plants and subsequent T1 progeny to confirm that the above-introduced DNA is present as a single copy using standard Southern analysis methods (see Maniatis). The phenotype imparted by the above DNA elements to the “Target” plants is FLP recombinase activity, Cre recombinase activity, and bialaphos resistance (FLP⁺,Cre⁺, BLP^{r}).

In a separate transformation experiment, inbred PHN46 is transformed using Agrobacterium, introducing the following “Donor DNA”:
Rb-(35S Pro-bar-pinII)—FRT1-GAT-pinII—pinII-moCah-loxP—Lb

After the recovery period on non-selective medium, calli are selected on bialaphos-containing medium and regenerated to produce “Donor” plants, with the phenotype of bialaphos resistance (BLP^{r}). The GATand moCah sequences have no promoters and thus are not expressed in the donor plants.

Target and donor plants are grown and upon reaching maturity are crossed to each other. In this scenario, the cassette from the donor containing inactive GAT and moCah sequences is removed from the donor locus and inserted into the target locus, in the process positioning the GAT sequence behind the Ubiquitin promoter (Ubi Pro) and the moCah sequence behind the Actin promoter (Actin Pro). The resultant functional orientation of these two structural sequences relative to the promoters results in expression of GAT and moCah and confers resistance to the herbicides Glyphosate (GLY^{r}) and Cyanimide (CYA^{r}), respectively. Thus, progeny seed from the above “Target×Donor” are planted and the resultant seedlings are sprayed with both herbicides.

Progeny in which proper recombinase-mediated cassette exchange has occurred (recombination at both the FRT1 and loxP sites) are readily identified (phenotype FLP⁻, BLP^{r}, GLY^{r} and CYA^{r}). The two new herbicide resistance traits (GLY^{r} and CYA^{r}) that resulted from the cassette exchange in the target locus will continue to co-segregate along with any other DNA elements originally introduced into the target locus adjacent but outside the FRT sites (i.e. they behave as a linkage group), and the inactivated FLP along with the two copies of 35S::bar:pinII that are now located at the donor locus will also segregate as a single unit. Thus, these two loci can easily be segregated away from each other in the next generation, and Glyphosate/Cyanamide double-resistant plants are obtained. PCR analysis across the recombined FRT1 and loxP junctions, as well as Southern analysis and sequencing can be used to confirm that precise recombination mediated by FLP recombinase occurred during the cassette exchange.

### EXAMPLE 4

### Recombinase-Mediated Cassette Exchange Results in Activation of One Marker Gene in the Target Locus and One Marker Gene in the Donor Locus

Jack, a Glycine max (I.) Merrill cultivar is transformed using particle bombardment, introducing the following “Target DNA”:
SCP1 Pro:FRT1:FLP.:pinII-CAMV35S Pro::HYG::nos Term:Kti3 Pro-FRT6

Calli are selected on hygromycin-containing medium and regenerated to produce “Target” plants. DNA is extracted from regenerated T0 plants and subsequent T1 progeny to confirm that the above-introduced DNA is present as a single copy using standard Southern analysis methods (see Maniatus). The phenotype imparted by the above DNA elements to the “Target” plants is FLP recombinase activity and hygromycin resistance (FLP⁺, HYG^{r}).

The progeny of the “Target” plants (FLP⁺, HYG^{r}) are then transformed using particle bombardment, introducing the following “Donor DNA” contained in a vector:
CaMV35S Term:FRT1:Gm-Als:: Gm-Als Term:35S Pro::GUS::Nos Term:FRT6:AmCyan1::Kti3 Term

The recombination between the FRT1 and FRT6 sites would occur resulting in the following sequences.

Recombined Target:

SCP1 Pro:FRT1:Gm-Als::Gm-Als Term:35S Pro::GUS::Nos Term:FRT6

Recombined Donor:

CaMV35S Term:FRT1:FLP::pinII-CAMV35S Pro::HYG::Nos Term:Kti3 Pro-FRT6:AmCyan1::Kti3 Term

After bombardment the calli are placed on media containing chlorsulfuron. The expression of Gm-Als confers resistance to herbicides that act to inhibit the action of acetolactate synthase (ALS), in particular sulfonylurea type herbicides such as chlorsulfuron. Cells containing sound integrations at both the 5′ and 3′ ends will be selected for by selecting for calli expressing resistance to chlorsulfuron and screening for calli resulting in a positive GUS assay. The expression of GUS can be determined by a histochemical assay (Jefferson, R. A. et al. EMBO Journal 6:3901-3907). The recombined donor cassette will express AmCyan1 because the sequence now has a promoter. Any cells undergoing recombination will transiently express Am-Cyan1 because the AmCyan1 sequence is positioned after the Kti3 promoter (Kti3 Pro). Expression of AmCyan1 allows the screening for blue fluorescence. Calli containing the recombined target sequence are grown into plants.

### EXAMPLE 5

### Recombinase-Mediated Cassette Exchange Results in Activation of Two Marker Genes in the Target Locus

Immature embryos from Hi-II corn were isolated. The immature embryos were transformed by infecting with Agrobacterium comprising the following construct.
Rb—Ubi Pro-FRT1-YFP-PinII Term-Ubi Pro-GAT-PinII Term-IN2-1 Term-GUS-FRT87-Actin Pro-Lb.

Starting at the right border the Ubiquitin promoter drives the yellow fluorescence protein coding region. The second Ubiquitin promoter drives the GAT coding region. The GUS coding region is positioned in the opposite direction and is driven by the Actin promoter.

After Agro-infection the embryos were placed on selection media containing glyphosate. The phenotype of the transformed cells was YFP+, GLY^{R}, GUS+. Cells growing on selection media that were also observed to be expressing YFP and GUS were regenerated to be used as target plants.

The target plants were grown and crossed to non-transgenic Hi-lI plants. The resulting immature embryos were isolated. The embryos expressing YFP were transformed using particle bombardment. Two plasmids were used, one carried the donor cassette and one carried the recombinase gene. The donor DNA comprised the following.
Rb-FRT1-BAR-PinII Term-Ubi Pro-Luciferase-PinII Term-N2-1 Term-CFP-FRT87-Lb

The plasmid carrying the recombinase contained Ubi::FLP::pinII. This plasmid was used at a lower DNA concentration so that it contributed enough transient recombinase activity for recombination to occur but was not readily incorporated into the genome. The Ubi::FLP::pinII may have infrequently randomly integrated into the genome. When this type of random integration occurs the construct can be removed through out-crossing.

After bombardment the immature embryos were placed on selection media containing bialophos in order to select for the expression of BAR. Cells growing on the selection media were also observed to express the Luc and CFP genes. Cells cultures expressing all three genes were analyzed using PCR. Analysis revealed that proper recombination occurred at the FRT1 and FRT5 junctions; the PCR products were the expected sequence lengths.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for obtaining a genetically modified plant cell said method comprising:
a) providing a plant cell stably transformed with a first polynucleotide comprising at least one recombinase recognition site and at least two recombinase-mediated gene trap elements;
b) introducing into said plant cell a second polynucleotide comprising at least one recombinase recognition site corresponding to the recombinase recognition site of the first polynucleotide and at least two recombinase-mediated gene trap elements;
c) providing a recombinase; and
d) identifying a genetically modified plant cell comprising a recombinase-mediated exchange between the first polynucleotide and the second polynucleotide.

2. The method of claim 1 wherein the second polynucleotide is introduced into the plant cell using pollination.

3. The method of claim 1 wherein the second polynucleotide is introduced into the plant cell using transformation techniques.

4. The method of claim 1 wherein the first polynucleotide comprises at least two recombinase recognition sites and the recombinase recognition sites are non-identical.

5. The genetically modified plant cell produced by the method of claim 1.

6. The genetically modified plant cell of claim 5 wherein said genetically modified plant cell comprises two stably integrated recombinase-mediated gene traps.

7. A plant comprising the genetically modified plant cell of claim 5.

8. The plant of claim 7 wherein the recombinase-mediated gene trap results in expression of a transgene that encodes a protein, said protein resulting in a phenotype that can be detected.

9. The plant of claim 7 wherein at least one of the recombinase-mediated gene traps encodes a selectable marker, screenable marker, or scorable marker.

10. A method for obtaining a genetically modified plant cell said method comprising:
a) obtaining a plant cell stably transformed with a first polynucleotide comprising two recombinase recognition sites and at least two recombinase-mediated gene trap elements;
b) introducing into said plant cell a second polynucleotide comprising two recombinase recognition sites corresponding to the two non-identical recombinase recognition sites of the first polynucleotide and at least two recombinase-mediated gene trap elements;
c) having active recombinase present during said introduction; and
d) identifying a genetically modified plant cell comprising a recombinase-mediated exchange between the first polynucleotide and the second polynucleotide at the chromosomal location of the first polynucleotide.

11. The method of claim 10 wherein the second polynucleotide is introduced into the plant cell using pollination.

12. The method of claim 10 wherein the second polynucleotide is introduced into the plant cell using transformation techniques.

13. The method of claim 10 wherein the plant cell is from a monocot plant or a dicot plant.

14. The method of claim 10 wherein said non-identical recombination sites are substrates for any combination of two different recombinases.

15. The method of claim 10 wherein said non-identical recombinase recognition sites are selected from the group consisting of a FRT, a mutant FRT, a LOX and a mutant LOX site.

16. The method of claim 10 wherein said recombinase is selected from the group consisting of a FLP, Cre, a codon optimized FLP, and a codon optimized Cre.

17. The genetically modified plant cell produced by the method of claim 10.

18. The genetically modified plant cell of claim 17 wherein said genetically modified plant cell comprises two stably integrated recombinase-mediated gene traps.

19. A plant comprising the genetically modified plant cell of claim 17.

20. The plant of claim 19 wherein the recombinase-mediated gene trap results in expression of a transgene that encodes a protein, said protein resulting in a phenotype that can be detected.

21. The plant of claim 19 wherein at least one of the recombinase-mediated gene traps encodes a selectable marker, a screenable marker, or a scorable marker.

22. A method for obtaining a genetically modified plant cell said method comprising:
a) obtaining a first plant cell stably transformed with a first polynucleotide, said first polynucleotide comprising two recombinase recognition sites and at least two recombinase-mediated gene trap elements;
b) growing said first plant cell into a first plant;
c) obtaining a second plant cell stably transformed with a second polynucleotide, said second polynucleotide comprising two recombinase recognition sites and at least two recombinase mediated-gene trap elements;
d) growing said second plant cell into a second plant;
e) crossing said first plant and said second plant together;
f) having active recombinase present during said crossing; and
g) identifying a genetically modified plant cell comprising a recombinase-mediated integration of the second polynucleotide at the chromosomal location of the first polynucleotide and also comprising two recombinase-mediated gene traps.

23. A method for obtaining a genetically modified plant cell said method comprising:
a) providing a plant cell stably transformed with a first polynucleotide comprising at least one recombinase recognition site, a first gene trap element, and a second gene trap element;
b) introducing into said plant cell a second polynucleotide comprising at least one recombinase recognition site, a third gene trap element, and a fourth gene trap element;
c) providing a recombinase; and
d) identifying a recombinase-mediated exchange between the first polynucleotide and the second polynucleotide in said plant cell, wherein said recombinase-mediated exchange results in the first polynucleotide comprising a first recombinase-mediated gene trap region and a second recombinase-mediated gene trap region, the first recombinase-mediated gene trap region comprising the first gene trap element and the third gene trap element in an orientation resulting in a first regulatory sequence operably linked to a first polynucleotide of interest, and the second recombinase-mediated gene trap region comprising the second gene trap element and the fourth gene trap element in an orientation resulting in a second regulatory sequence operably linked to a second polynucleotide of interest.

24. A method for obtaining a genetically modified plant cell said method comprising:
a) providing a plant cell stably transformed with a first polynucleotide comprising at least one recombinase recognition site, a first gene trap element, and a second gene trap element;
b) introducing into said plant cell a second polynucleotide comprising at least one recombinase recognition site, a third gene trap element, and a fourth gene trap element;
c) providing a recombinase; and
d) identifying a recombinase-mediated exchange between the first polynucleotide and the second polynucleotide in said plant cell, wherein said recombinase-mediated exchange results in the first polynucleotide comprising a first recombinase-mediated gene trap region and in the second polynucleotide comprising a second recombinase-mediated gene trap region, the first recombinase-mediated gene trap region comprising the first gene trap element and the third gene trap element in an orientation resulting in a first regulatory sequence operably linked to a first polynucleotide of interest, and the second recombinase-mediated gene trap region comprising the second gene trap element and the fourth gene trap element in an orientation resulting in a second regulatory sequence operably linked to a second polynucleotide of interest.
